(19) United States
(12) Patent Application Publication
(10) Pub. No.: US 2004/0053361 A1
(43) Pub. Date: Mar. 18, 2004

(54) Nucleic acid construct useful for expressing transgenes in particular in embryonic stem cells

(76) Inventors: Ludovic Vallier, Lyon (FR); Jimmy Mancip, Lyon (FR); Daniel Metzger, Strasbourg (FR); Pierre Chambon, Blaesheim (FR); Jacques Samarut, Villeurbanne (FR); Pierre Savatier, Lyon (FR)

Correspondence Address: YOUNG & THOMPSON, 745 SOUTH 23RD STREET 2ND FLOOR, ARLINGTON, VA, 22202

(21) Appl. No.: 10/297,475

(22) PCT Filed: Jun. 7, 2001
(86) PCT No.: PCT/IB01/00996

(30) Foreign Application Priority Data
Jun. 7, 2000 (FR) 0007298

(51) Int. Cl. C12P 21/02; C12N 5/06; C07H 21/04; C12N 9/22

(52) U.S. Cl. 435/69.1; 435/199; 435/320.1; 435/325; 536/23.2

(57) ABSTRACT

A promoter-free nucleic acid construct, includes a selection sequence and a sequence coding for a protein of interest distinct from the selection sequence, the coding sequence being preceded upstream by a sequence enabling its translation by ribosomes and the uses of the construct in the fields of biology and medicine.

The invention relates to a nucleic acid construct useful for expressing transgenes in particular in embryonic stem cells.

The culture of embryonic stem cells (also called ES cells) has opened the way to numerous applications in the field of biology and medicine: cell and tissue transplants in the context of a so-called “regenerative” medicine, production of animal models from these cells for studying pathologies and the development of medicaments, and the like.

These cells may be genetically modified in vitro and then reimplanted into a recipient embryo in order to obtain a transgenic animal.

In particular, genes may be mutated or deleted by homologous recombination. However, the inactivation of a gene, while it is essential for the development of the embryo, will most often cause termination of this development.

To overcome these problems, inducible recombination systems have been developed. Feil et al. (1996) and Zhang et al. (1996) have in particular proposed using a vector comprising the Cre recombinase sequence fused to an estrogen binding domain mutated so as to bind solely to tamoxifen, the whole being under the control of a strong promoter (CMV promoter). However, the results obtained by Zhang et al. are disappointing both in terms of the background expression and the percentage induction. Thus, the induction of recombinase occurs in less than 60% of the cells while the background expression increases constantly over time. After 8 weeks of culture, all the cells are induced in the absence of an inducer.

The functional information provided by gene invalidation is sometimes supplemented by another technique which consists, by contrast, in overexpressing a gene. This experimental strategy is generally used by producing transgenic animals (such as mice) using the microinjection of an expression plasmid into the oocytes. However, as in the case of gene invalidation, the production of a transgenic animal for a given gene requires that its expression remains compatible with a harmonious embryonic development.

In the opposite case, the development of the transgenic embryos is interrupted. Several experimental strategies using either promoters whose activities are tissue- or organ-specific, or inducible expression systems (system inducible by zinc, by tetracycline or by doxycycline), have been developed. However, the use of these inducible expression systems is cumbersome given the low efficiency of producing transgenic animals by the technique of microinjection of DNA into the oocyte. In addition, the “random” integration of the transgenes into the genome of the oocyte often compromises their expression according to the criteria required by the experimenter.

In parallel, an approach termed “promoter-trap” had been envisaged for identifying and mutating developmental genes in mice (Friedrich and Soriano, 1991).

According to this approach, the expression of a reporter gene was initiated with the aid of an endogenous promoter, the reporter gene itself not having its own promoter. This approach was adopted, for example, in U.S. Pat. No. 5,922,601 or patent application WO 98/14 614, still in order to identify and mutate genes present in the genome of the cells.

Moreover, incidentally, a promoter-free vector, intended for the expression of a tetracycline-dependent transactivator tTA, has been described by Böger and Gruss, 1999.

The authors of the present invention have now developed an expression system which solves all the problems mentioned above.

The subject of the invention is more precisely a nucleic acid construct with at least two coding sequences, one for selection, the other encoding a protein of interest. This construct comprises:

i) a splice acceptor site at the 5′ position,

ii) a selection sequence, optionally preceded upstream by a sequence allowing its translation by the ribosomes,

iii) a sequence encoding a protein of interest distinct from the selection sequence, said coding sequence being preceded upstream by a sequence allowing its translation by the ribosomes,

iv) a transcription termination sequence at the 3′ position,

said construct being free of any promoter for transcription of said selection sequence or of said sequence encoding a protein of interest,

it being in addition understood that said protein of interest is not the transactivator protein tTA.

The expression “nucleic acid construct” is understood to mean in particular a nucleic acid such as linear or circular DNA or RNA.

The nucleic acid construct of the invention may comprise from upstream to downstream, the splice acceptor site, the selection sequence, optionally preceded by a sequence allowing its translation by the ribosomes, the sequence encoding a protein of interest, preceded by a sequence allowing its translation by the ribosomes, and the transcription termination sequence. This type of construct is preferred.

Alternatively, the nucleic acid construct of the invention may however comprise, from upstream to downstream, the splice acceptor site, the sequence encoding a protein of interest, preceded by a sequence allowing its translation by the ribosomes, the selection sequence, preferably preceded by a sequence allowing its translation by the ribosomes, and the transcription termination sequence.

The expression “sequence allowing translation by the ribosomes” is understood to mean, for example, an IRES sequence (internal ribosome entry site). It may be in particular a mammalian IRES sequence, such as the internal ribosome entry site of the gene encoding the protein GRP79, also called Bip, which binds the heavy immunoglobulin chain. It is also possible to use a IRES sequence of picornaviruses, such as the IRES sequence of the encephalomyocarditis virus (EMCV), (Jackson et al., 1990; Kaminski et al., 1990), preferably nucleotides 163 to 746 of this sequence, of the poliovirus, preferably nucleotides 18 to 640, or of the foot and mouth disease virus (FMDV), preferably nucleotides 369 to 804. It is also possible to use IRESs derived from retroviruses such as the Moloney murine virus (MoMLV).

It is also possible, moreover, to use any sequence allowing the translation of several proteins from a single mRNA whose transcription is initiated by a single promoter. For example, these sequences may simply allow continuity of the ribosome reading between two cistrons encoding two distinct proteins.

The expression “selection sequence” is understood to mean a sequence which makes it possible to sort the cells which have integrated the nucleic acid construct of the invention and those in which the transfection has failed.

These selection sequences may be “positive” or “negative” and dominant or recessive. A “positive” selection sequence refers to a gene encoding a product which allows only the cells carrying this gene to survive and/or to multiply under certain conditions. Among these “positive” selection sequences, there may be mentioned in particular the sequences of genes for resistance to an antibiotic, such as for example neomycin (neo^{r}), hygromycin, puromycin, zeoycin, blasticidin or phleomycin. Another possible selection sequence is hypoxanthine phosphoribosyl transferase (HPRT). Cells which carry the HPRT gene can grow on HAT medium (containing aminopterin, hypoxanthine and thymidine), while the HPRT-negative cells die on the HAT medium.

Conversely, a “negative” selection sequence refers to a gene encoding a product which may be induced to selectively kill the cells carrying the gene. Nonlimiting examples of this type of selection sequences include the thymidine kinase of the herpes simplex virus (HSV-tk) and HPRT. Cells which carry the HSV-tk gene are killed in the presence of gancyclovir or FIAU (1(1,2-desoxy-2-fluoro-β-D-rabinofuranosyl)-5-iodouracil). Cells which carry the HPRT gene can be selectively killed by 6-thioguanine (6TG).

Other examples of “positive” or “negative” selection sequences are well known to persons skilled in the art.

Advantageously, the nucleic acid construct of the invention may also comprise a detection sequence.

The expression “detection sequence” is understood to mean a sequence encoding a detectable protein, useful as a marker for easily evaluating the level of expression of the protein of interest. The expression “reporter gene” is also used in this case. It may be, for example, a sequence encoding an enzyme such as β-galactosidase (β-GAL), alcohol dehydrogenase (ADH), alkaline phosphatase such as human Alkaline Phosphatase (Aph), green fluorescent protein (GFP), and chloramphenicol acetyltransferase (CAT), luciferase, or any other detectable marker well known to persons skilled in the art.

Preferably, said detection sequence may be coupled to the selection sequence. This coupling may be performed via a sequence allowing translation by the ribosomes, as defined above, or by fusion between the detectable sequence and the selection sequence. It is possible in particular to use the βgeo element which encodes the fusion protein β-galactosidase-neo^{r} (Friedrich and Soriano, 1991).

The expression “transcription termination sequence” is understood to mean any sequence which makes it possible to stop the transcription, in particular a STOP site contained in a polyadenylation (polyA) sequence. It may be a virus-derived polyA, in particular the “Simian Virus 40” (SV40) polyA, or a polyA derived from a eukaryotic gene, in particular the polyA of the gene encoding Phosphoglycerate Kinase (pgk-1), or the polyA of the gene encoding rabbit β-globin.

According to a first embodiment of the invention, said protein of interest may be an inducible recombinase. Preferably, it is possible to use the bacteriophage P1 Cre recombinase (Abremski et al., 1983), or for example the yeast Flp recombinase (Logie et al., 1995). These recombinases are modified or operably linked (in particular by fusion) to a sequence providing them with the induction property. It is thus possible to use a recombinase fused to the ligand-binding domain of the estrogen receptor (ER), which receptor has been mutated beforehand so as to no longer bind endogenous estrogens. On the other hand, it can be activated by tamoxifen or by one of its analogs (Feil et al., 1996). It is also possible to use a recombinase fused to other domains such as the ligand binding domain of the progesterone receptor (PR) (Kellendonk et al., 1996) or the ligand binding domain of the glucocorticoid receptor (GR) (Brocard et al., 1998). These domains are mutated beforehand so as to no longer be activated by their natural ligand but only by synthetic molecules such as dexamethasone or RU486.

Advantageously, it is possible to use the Cre ER^{T2} sequence (Feil et al., 1997) which is a sequence encoding a protein whose recombinase activity is very easily inducible by tamoxifen or by its analogs.

More particularly, the present invention provides a nucleic acid construct as defined above, comprising from upstream to downstream:

i) a splice acceptor site,

ii) a neomycin resistance selection sequence, preceded upstream by a detectable sequence, encoding β-galactosidase, the whole being designated β-geo,

iii) a Cre-ER^{T2} sequence, preceded upstream by an IRES sequence allowing its translation by the ribosomes,

iv) at least one polyA sequence containing at least one STOP site, for termination of transcription.

According to a second embodiment of the invention, said protein of interest may be a protein of therapeutic interest or a differentiation factor. It is possible to mention in particular, as protein of interest, blood proteins, hormones, growth factors, cytokines, neurotransmitters, enzymes, antibodies, factors involved in DNA repair, DNA structural proteins, transcription factors, transcription coactivators or corepressors, proteins of the HLA system, proteins of the immune system, membrane receptors, proteins involved in cell division, oncogenes, tumor suppressors, hormone receptors, factors involved in programmed cell death, proteins involved in cell migration, cytoskeletal proteins, viral proteins, proteins derived from a prokaryotic organism, and the like.

According to a third embodiment of the invention, it is possible to replace said sequence encoding a protein of interest by an antisense sequence, so as to block the translation of a protein of interest.

The subject of the invention is also a nucleic acid construct as defined above, in which recombinase recognition sequences such as the LoxP sequences, surround the cassette formed by said selection sequence, optionally preceded upstream by a sequence allowing its translation by the ribosomes, and followed downstream by at least one additional transcription termination sequence, said cassette being placed upstream of said sequence encoding the protein of interest.

In this case, said protein of interest may be a detectable marker protein (useful in the context of research protocols), or advantageously a protein of therapeutic interest or a differentiation factor.

A detection sequence, encoding a detectable marker protein, and optionally preceded by a sequence allowing its translation by the ribosomes, may then be inserted into said cassette.

This detection sequence, like the selection sequence, is not under the control of any promoter in the nucleic acid construct of the invention.

One subject of the invention relates more particularly to a nucleic acid construct as defined above, comprising from upstream to downstream:

i) a splice acceptor site,

ii) a cassette formed from upstream to downstream by

optionally one sequence, such as an IRES sequence, allowing translation, by the ribosomes, of the selection sequence which follows,

a selection sequence, such as a sequence for resistance to hygromycin,

optionally a sequence encoding a detectable marker protein, such as a sequence encoding human alkaline phosphatase (Aph), preceded by a sequence, allowing its translation by the ribosomes,

a transcription termination sequence comprising several STOP sites in several polyAs,

said cassette being surrounded by LoxP sequences,

iii) a sequence encoding a protein of interest, said coding sequence being preceded upstream by a sequence, such as an IRES sequence, allowing its translation by the ribosomes,

iv) a transcription termination sequence.

The subject of the invention is also a vector into which a nucleic acid construct as defined above is inserted.

It may be a plasmid vector of bacterial origin or a recombinant viral vector, such as a modified adenovirus or retrovirus vector such as the vector ROSA β GEO (Friedrich et al., 1991). Advantageously, it is possible to use the bacterial plasmid pBSK or the bacterial plasmid pIresHyg (Clontech reference 6061).

The subject of the invention is also a host cell into which at least one such vector has been stably transferred.

The term “host cell” comprises any mammalian cell or another eukaryotic cell, in culture or in vivo, as part of an organism, it being possible for said cell to be fused or genetically modified beforehand. It may be for example ES cells (embryonic stem cells), EG cell lines (embryonic germ cells), tetracarcinoma stem cell lines such as F9 cells, immortalized fibroblast lines such as NIH 3T3, lymphoblastic cell lines such as Jurkat cells, and the like.

According to a particular embodiment of the invention, there are transferred into the host cell at least one nucleic acid construct as defined above comprising a sequence encoding an inducible recombinase, and at least one nucleic acid construct comprising sequences for recognition of said recombinase and a sequence encoding a protein of interest, such that these two constructs are cointegrated into the genome of said cell.

The transfer of the vector into the host cell may be carried out by means of standard techniques known to persons skilled in the art, for example by electroporation, calcium phosphate precipitation (Sambrook et al., 1989), or lipofection.

In general, the nucleotide vector of the invention may be released in naked form, that is to say free of any agent facilitating the transfection, or in combination with such an agent, whether it is for example a chemical agent which modifies cell permeability (such as bupivacaine), liposomes, cationic lipids or microparticles, for example, of gold, silica or tungsten.

The mode of transfer chosen depends mainly on the host cell, as is well known to the person skilled in the art.

More particularly, the present invention relates to the case where the host cell is a stem cell, preferably an embryonic stem cell (ES cell).

ES cells are cells obtained from the cellular mass constituting an embryo at the blastocyst stage. They are capable of undergoing differentiation into all the cell types of an adult organism, in particular into germ cells. These cells may be cultured so as to develop cell populations which are totipotent, that is to say capable of giving all the possible types of differentiated cells, or pluripotent, that is to say capable of giving certain types of cell lines (in particular hematopoietic cells), or which are differentiated or undergoing differentiation, according to the culture conditions chosen (Fraichard et al., 1995; Takahashi et al., 2000; Reubinoff et al., 2000).

The ES cells of the invention may be human cells or may be derived from a nonhuman animal, preferably a mammal.

The subject of the invention is also a bank of cell lines obtained from host cells as defined above, into the genome of which said vector(s) as defined above has (have) become functionally integrated.

In particular, the authors of the invention have developed a bank of ES cell lines which have functionally integrated into their genome a vector as described above allowing the expression of a tamoxifen-inducible Cre recombinase such as Cre-ER^{T2}. They selected the ES cell lines characterized by three criteria:

Each of these lines possesses a very high level of expression of the inducible recombinase.

No recombinase activity is detectable in these lines in the absence of tamoxifen or of one of its derivatives. On the other hand, the recombinase activity of Cre is easily induced by tamoxifen or one of its derivatives.

The expression of the inducible recombinase is stable during embyronic development, but it may be either ubiquitous or tissue specific.

The cells thus characterized are used to construct novel banks of ES cell lines into whose genome is integrated a second vector as described above allowing the expression of a protein of interest by the inducible Cre recombinase.

The ES cells derived from these various banks and therefore carrying the nucleic acid constructs of the invention, may be used to obtain genetically modified animals (also called transgenic animals). The techniques conventionally used to obtain transgenic animals from ES cells are the technique of injection into the blastocyst and the aggregation technique (Hogan et al., 1994, Manipulating the Mouse Embryo, Cold Spring Harbor Laboratory Press). These techniques consist in injecting the ES cells into a recipient embryo at the blastocyst stage (technique of injection into the blastocyst) or in aggregating the ES cells with a recipient embryo at the morula stage (aggregation technique). The chimeric embryos obtained are reimplanted into the uterus of a carrier female. The chimeric animals obtained consist of a mixture of wild-type cells and of cells carrying the genetic modification. To obtain transgenic animals, the chimeric mice should then be crossed with wild-type mice. Fertilization occurs either with a wild-type cell, or with a genetically modified cell.

Thus, the invention provides means for generating transgenic animals capable of inducibly or noninducibly overexpressing a protein of interest. The use of the nucleic acid vectors as described above in the ES cells has, in addition, numerous advantages compared with the technique of microinjection of DNA into the oocyte.

With the technique of microinjection into the oocyte, the insertion of the transgene into the recipient genome occurs randomly and without any means of selection. Thus, its expression is influenced, in general negatively, by the chromosomal environment of the site of insertion. This negative influence often translates into an extinction of expression or a mosaicism of this expression. In numerous cases, the level of expression of the transgene proves to be insufficient, or the activity of the promoter which controls its expression is disrupted by endogenous regulatory elements which modify the tissue-specificity thereof. By contrast, the application of the system of the invention to ES cells allows the experimenter to select in vitro the ES cell line which makes it possible to obtain a transgenic animal overexpressing the protein of interest in the expected tissues. The selection of the clones is carried out a posteriori according to the level of expression and of the domains of expression of the protein of interest.

The absence of a promoter from the vectors as described above implies that their function is strictly dependent on the site of the host genome into which they become inserted. The vectors of the invention appropriate the properties of natural expression of the site into which they become integrated. This capacity makes it possible to considerably reduce all the problems of extinction of expression and of mosaicism which are frequent with the expression systems conventionally used like the viral promoters: Cyto-Megalo virus (CMV) promoter SV40 promoter.

The system of the invention therefore provides simpler and more cost-effective means for generating transgenic animals.

The subject of the present invention is therefore nonhuman transgenic animals capable of being obtained from an ES cell as defined above.

The transgenic animals thus generated may be particularly useful for producing recombinant proteins of interest, such as proteins of therapeutic interest cited above. It is also possible to cause these animals to overexpress functionally defective proteins, like recombinant proteins of interest. The transgenic animals obtained are then useful as experimental models of pathologies caused by the expression of these nonfunctional proteins, for example truncated or muted proteins. That is the case in particular of the CFTR (cystic fibrosis transmembrane regulator) protein whose mutation is responsible for cystic fibrosis in human beings. It is also possible to cause these animals to overexpress certain oncogenes like ras, jun, fos or β-catenin proteins. Likewise, it is possible to express negative dominants of certain anti-oncogenic proteins like p53 or pRb.

The ES cells incorporating the nucleic acid constructs of the invention may also be exploited in the context of a cell transplantation strategy.

The general principle of this strategy is based on the in vitro differentiation of ES cells into neural or hematopoietic stem cells, and the like, and then injecting these “predetermined” cells into an animal or a human. Tissue repair is also spoken of in this regard (Watt and Hogan, Science, 2000).

The invention therefore extends to a method for preparing differentiated cells, in which totipotent ES cells as mentioned above are cultured in the presence of differentiation agents and, where appropriate, a recombinase inducing agent.

The expression system inducible with a recombinase, as described above, makes it possible more particularly to induce the controlled expression of genes whose activity is responsible for placing the stem cell in a specific differentiation pathway.

Said “differentiation agents” are well known to a person skilled in the art. There may be mentioned in particular retinoic acid (RA) (Renoncourt et al., 1998), dimethyl sulfoxide (DMSO) and gamma-aminobutyric acid (GABA) (Dinsmore et al., 1996).

Likewise, the conditions for culturing ES cells are now well established (Dinsmore et al., 1998; Dinsmore et al., 1996).

Also included in the invention are the cells which can be obtained by this method.

The differentiated cells thus obtained can serve as a cellular model for replacing animal experimentation. Indeed, the method described above makes it possible to produce a large quantity of differentiated cells in a given cell type. It is then possible to easily test the toxicity of molecules with therapeutic potential on these cells instead of testing it directly on animals.

The subject of the invention is also a method of therapeutic treatment in which cells modified and differentiated in vitro beforehand are implanted in a recipient organism requiring such a treatment.

This cell transplantation technology using predetermined cells or cells differentiated in vitro from ES cells then renders possible strategies for very specific metabolic corrections. For example, in the treatment of neurodegenerative diseases, the transplantation of neuromediator producing neuronal cells is of obvious clinical interest. Using in particular the expression system inducible in ES cells, it is possible to introduce an inactive gene encoding a neurotransmitter or stimulating its synthesis, and then to induce the differentiation of the ES cells into neural cells, and to activate the expression of the gene at the time of reimplanting the cells into the recipient organism.

In general, the invention also relates to a method of therapeutic treatment in which there are implanted into a recipient organism requiring such a treatment, cells modified and differentiated in vitro beforehand, integrating a nucleic acid construct which comprises a sequence encoding an inducible recombinase, and a nucleic acid construct of interest and recombinase recognition sequences, and a quantity of inducing agent sufficient to allow the expression of the protein of interest is administered to said recipient organism.

The subject of the invention is also an in vitro method for producing recombinant proteins of interest, in which there are cultured ES cells into whose genome there has been integrated a nucleic acid construct as defined above comprising a sequence encoding a recombinant protein of interest, under conditions allowing the expression of said protein of interest, and the protein thus produced is recovered.

According to a particular embodiment of the invention, the ES cells used are embryonic stem cells, into whose genome there are cointegrated at least one nucleic acid construct as defined above comprising a sequence encoding an inducible recombinase and at least one nucleic acid construct comprising sequences for recognition of said recombinase, and a sequence encoding a protein of interest, said cells being cultured in the presence of differentiation agents, the differentiated cells thus obtained then being brought into contact with an agent inducing said recombinase, so as to allow the expression of said protein of interest.

This method is then particularly advantageous for producing a protein in vitro in the cell type which manufactures it naturally.

Indeed, to be functional, these molecules often require post-translational modifications which do not operate in the microorganisms customarily used for producing them, but which are sometimes only performed in the cell types which naturally make these molecules, which is the case for the above differentiated cells.

The invention also relates to the development of animal models for studying genes involved in a pathology or genes involved in differentiation processes.

The subject of the invention is more particularly a method for producing a nonhuman transgenic animal which is in particular useful as a model for studying genes involved in a pathology, in which a nonhuman animal, into whose genome has been integrated at least one nucleotide acid construct as defined above comprising a sequence encoding an inducible recombinase, is crossed with a nonhuman animal in whose genome a gene of interest is surrounded by two sites recognized by the inducible recombinase, so as to obtain a nonhuman transgenic animal which, when it is subjected to an agent inducing said recombinase, undergoes deletion of said gene of interest.

The gene surrounded by two sites recognized by the inducible recombinase is called “floxed” gene. The nonhuman animal possesses a wild-type phenotype, which suggests that the “floxed” gene is functional, the sites recognized by the inducible recombinase being introduced so as not to disrupt its function. Some of the animals derived from this crossing possess in their genome the two genetic modifications cited above. It is then possible to destroy the floxed gene by inducing the activity of the recombinase by an inducing agent. The animal thus acquires a mutant phenotype if the destroyed gene possesses an important function.

This method of deleting an inducible gene makes it possible to destroy any gene at any age of the animal, which is currently impossible with existing techniques.

The nonhuman transgenic animals, such as in particular mice or another animal cited above, which are capable of being obtained by this method, are also included in the invention.

The following examples and figures illustrate the invention without limiting the scope thereof.

### LEGEND TO THE FIGURES

FIG. 1 represents a diagram of the principle of the functioning of a vector according to the invention designed to overexpress the inducible recombinase Cre-ER^{T2}: the plasmid pGTEV-Cre-ER^{T2}.

FIG. 2 represents a restriction map of the vector pGTEV-Cre-ER^{T2}.

FIG. 3A represents a diagram of the vector pIGTE2-Aph, FIG. 3B represents a diagram of the vector pIGTE3, FIG. 3C represents a diagram of the vector pIGTE4, FIG. 3D represents a diagram of the vector pIGTE5.

FIG. 4 represents a restriction map of the vector pIGTE2-Aph.

FIG. 5 represents a diagram of the principle of the functioning of a vector according to the invention designed to inducibly overexpress Aph: the plasmid pIGTE2-Aph.

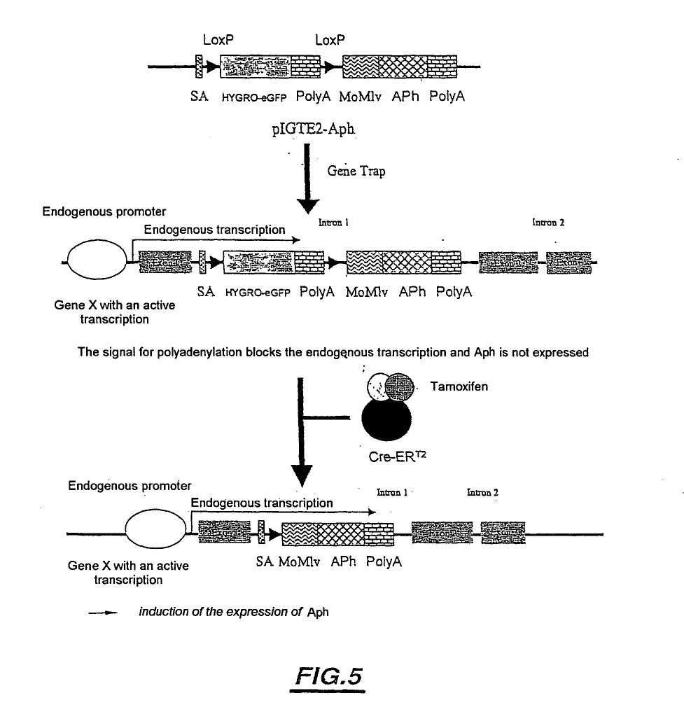

FIG. 6 is a photograph of 8.5-day old mouse transgenic embryos after fertilization. These embyros were obtained using ES Cre ER^{T2} cells which have integrated into their genome the vector pIGTE2-Aph. The Aph activity is detectable by histochemical staining. Thus, the cells which express Aph are stained brown while the cells which do not express Aph remain white. As may be seen in this figure, the embryos induced in vivo with hydroxytamoxifen (on the right and on the left) strongly express Aph in all the tissues whereas the negative control (embryo in the center) expresses Aph only in a few cells.

FIGS. 7A and 7B represent diagrams showing the induction of eGFP in the ES-Cre-ER^{T2} clones by hydroxytamoxifen (OHT). FIG. 7A gives the percentage of cells which express eGFP in the presence or in the absence of hydroxytamoxifen, while FIG. 7B highlights the level of induction.

FIG. 8A is a diagram representing the result of a test of induction of the Aph activity in various ES-Cre-ER^{T2}/pIGTE2-Aph mouse lines in the presence or in the absence of hydroxytamoxifen (OHT). FIG. 8B is a photograph representing an induction of the expression of β-galactosidase using a conventional expression system in ES cells.

FIG. 9 is a set of photographs representing ES-CreER^{T2}/pIGTE2-Aph cell lines after histochemical staining to detect β-galactosidase (FIG. 9A), after histochemical staining to detect the Aph activity, in the absence of hydroxytamoxifen (FIG. 9B), and after histochemical staining to detect the Aph activity in the presence of hydroxytamoxifen (FIG. 9C).

### EXAMPLES

### Example 1

### Construction of the Vector pGTEV-Cre ER^{T2}

The authors of the invention constructed a vector for expressing the recombinase Cre ER^{T2} inducible by tamoxifen (Feil et al., 1997). This vector does not contain a promoter.

Transcription may only be initiated from a cellular promoter situated near the site of integration. The splice acceptor site (SA) allows correct splicing of the messenger and the formation of a fusion protein when the integration occurred in an intron. The vector also expresses the fusion protein β-galactosidase-neo^{r}(βgeo gene). The expression of the Cre-ER^{T2} recombinase is coupled to that of β-galactosidase by virtue of the use of an IRES sequence (internal ribosome entry sequence).

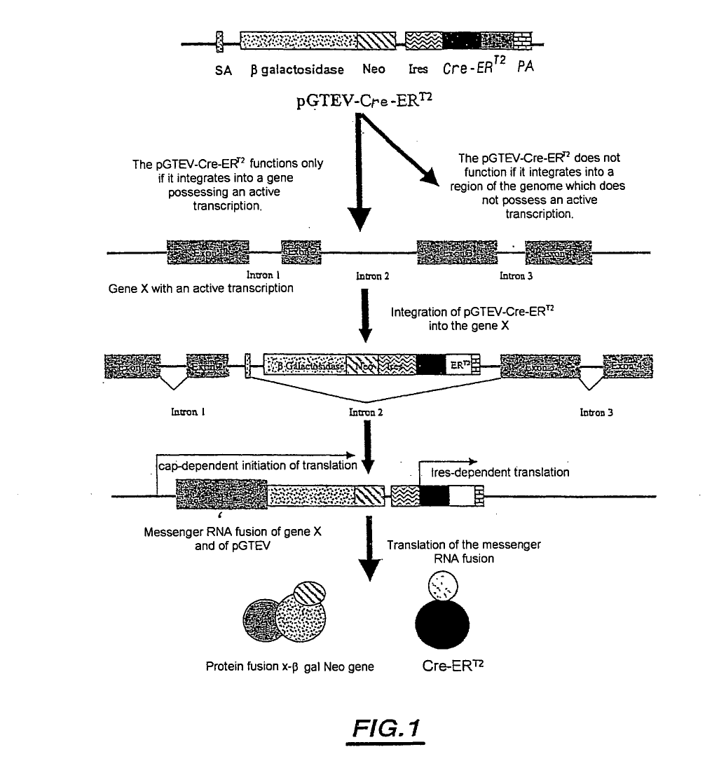

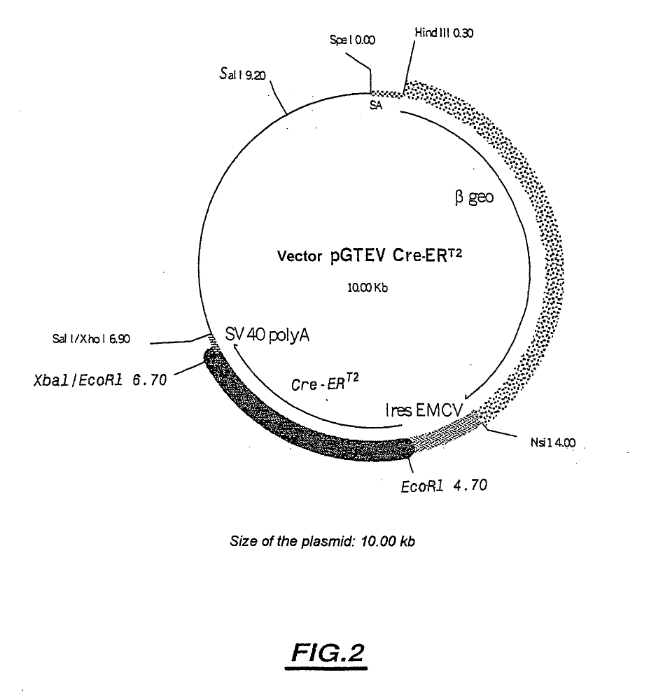

This pGTEV vector (cf FIGS. 1 and 2) was constructed as follows: the SA β geo sequence was amplified by PCR and produced from the vector ROSA β geo (Friedrich et al., 1991) using the following oligonucleotides 5′ AGA ACC AAT GCA TGC TGA TCA GCG AGG TTT A 3′ (SEQ ID No. 1) and 5′ AAG GAA AAA AGG GGG CGC CTA TGG CTC GTA CTC TAT AG 3′ (SEQ ID No. 2). The 3.7 kilobase (kb) fragment thus amplified was digested with the enzymes SpeI and NsiI and then introduced by cohesive ligation into the CMV Ires-Cre-ER^{T2} vector previously digested with the same enzymes. The CMV Ires-Cre-ER^{T2} vector was obtained by inserting the Cre ER^{T2} cassette obtained from the vector pCre-ER^{T2} (Feil et al., 1997) digested with EcoRI. The ends of the fragment thus obtained were made blunt using T4 DNA polymerase. The fragment was introduced by ligation into the vector pIresNeo (Clontech, catalog reference 6060-1) digested with SmaI and XbaI and whose ends were also made blunt using T4 DNA polymerase. The vector pGTEV is thus obtained.

### Example 2

Expression of the Cre-ER^{T2} Recombinase in Mouse ES Cells

2.1 Electroporation of the ES Cells:

The ES cells are subjected to a treatment with trypsine and then rinsed twice in GMEM medium. They are finally resuspended in GMEM at a concentration of 6.25×10⁶ cells/ml. For a stable expression, 40 μg of plasmid are digested with SspI and then added to an electroporation cuvette (Biorad) to 0.8 ml of the solution of ES cells. The cells are then subjected to electroporation at a voltage of 250 V for a capacitance of 500 μF. After electroporation, the cells are placed in previously irradiated feeder cells. 48 hours after electroporation, the cells are brought into contact with an antibiotic G418.

2.2. Evaluation of the Level of Expression of the Recombinase:

The cells which have integrated the vector near an active cellular promoter are resistant to G418 and synthesize β-galactosidase, which makes it possible to easily select them.

The production of recombinase being in addition proportional to that of β-galactosidase, the authors of the invention were able to easily evaluate the level of expression of the recombinase.

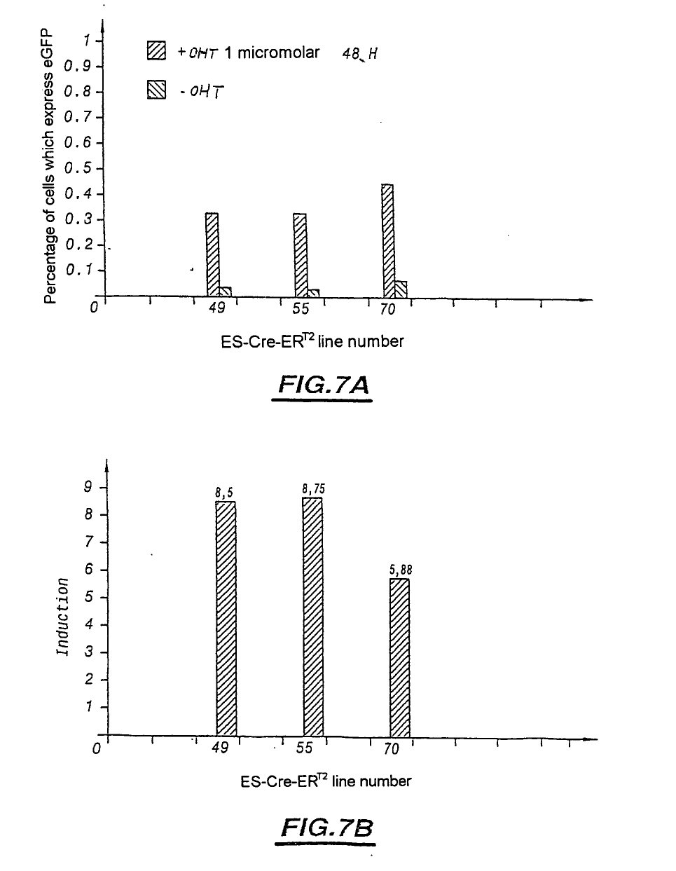

The use of this vector makes it possible to obtain a level of expression of β-galactosidase and of Cre-ER^{T2} recombinase which has never been achieved with the customary expression vectors. Such a level of expression is necessary in order to obtain a high recombinase activity in the presence of a nontoxic concentration of hydroxytamoxifen (FIGS. 7A and 7B).

### Example 3

Selection of ES-Cre-ER^{T2} Cell Lines

The authors of the invention made a bank of 110 ES cell lines, each element of the bank being characterized by a specific site of integration of the vector PGTEV-Cre ER^{T2}. The level of expression and the domains of expression of the Cre-ER^{T2} recombinase therefore vary for each line according to the site of integration of the vector. In a first instance, the authors of the invention selected lines characterized by a very high recombinase expression level so that the activity thereof is easily induced by hydroxytamoxifen. For that, the authors of the invention defined three criteria which make it possible to select the lines that are a priori the most efficient:

1) the Cre-ER^{T2} recombinase expression level should be very high, both in the ES cells and in the differentiated cells (differentiation induced in vitro by formation of embryoid bodies). The recombinase expression level was evaluated based on the β-galactosidase expression level (histochemical staining).

2) the Cre-ER^{T2} recombinase activity should be zero in the absence of hydroxytamoxifen (absence of background expression) and should be rapidly induced when hydroxytamoxifen is added to the culture medium. To evaluate this parameter, a reporter vector for the Cre-ER^{T2} recombinase activity was constructed. This vector, called pCAAG-loxP-STOP-loxP-ADH, comprises, from upstream to downstream, (1) a promoter CAAG which is a very powerful promoter functioning in the ES cells, (2) a gene for resistance to hygromycin and a polyadenylation (polyA) signal allowing transcription to be stopped, the whole formed by the resistance gene and the polyA sequence being surrounded by loxP sequences, (3) a sequence encoding alcohol dehydrogenase (ADH), which confers a gray color on the cells after histochemical staining, and, finally, (4) a polyA sequence.

The vector pCAAG-loxP-STOP-loxP-ADH was constructed as follows: the vector pPHCAAG-BstXI (Niwa et al., 1991) was digested with the enzymes SalI and XhoI. The fragment of 4 Kilobases thus obtained corresponds to the pCAAG promoter. This fragment was introduced by cohesive ligation into the vector pBSK previously digested with SalI. The novel vector obtained is called pBSK pCAGG. The vector pT102 was digested with the enzymes HindIII-NotI. The fragment thus obtained corresponds to a loxP-STOP-loxP cassette which was introduced by cohesive ligation into the vector pBSK pCAAG itself digested with the enzymes HindIII-NotI. The novel vector obtained is called pCAAG loxP-STOP-loxP. The vector pRc/CMV-ADH (Gautier et al., 1996) was digested with the enzyme BamHI. The ends of the fragment thus obtained were made blunt using T4 DNA polymerase. The fragment was introduced by ligation into the vector pCAAG loxP-STOP-loxP digested with NotI and whose ends were also made blunt using T4 DNA polymerase. The vector pCAAG-loxP-STOP-loxP-ADH is thus obtained.

The ADH reporter gene for alcohol dehydrogenase functions only if the Cre-ER^{T2} recombinase is active because a transcription stop signal surrounded by two loxP sites prevents its transcription. The activation of the recombinase by hydroxytamoxifen should cause the transcription stop signal to disappear by excision at the level of the loxP sites in order to allow the expression of the ADH reporter gene. The authors of the invention were thus able to select the lines meeting the two criteria defined above (zero recombinase activity in the absence of hydroxytamoxifen, maximum activity in the presence of hydroxytamoxifen).

3) The Cre-ER^{T2} recombinase expression should be conserved after differentiation in vivo into the developing embryos. The ES cell lines which fulfilled the first two criteria were injected into recipient embryos at the blastocyst stage (Hogan et al., 1994). The chimeric embryos thus obtained were reimplanted into the uterus of a carrier mouse, and then dissected at various stages of development in order to determine the domains of expression of β-galactosidase. For each of the lines tested, the ubiquitous and tissue character of the expression of the recombinase was thus able to be defined.

These three criteria made it possible to select 15 ES cell lines: (i) which allow the expression of recombinase in all the tissues of the embryo during the first stages of development; (ii) whose recombinase activity is very closely regulated by hydroxytamoxifen in vitro.

These lines, called ES-Cre-ER^{T2}, were able to be isolated using a novel type of vector, pGTEV-Cre ER^{T2}, which allows expression of the transgene at a very high level and with remarkable stability. It should be noted that the expression plasmids customarily used for overexpressing genes (plasmids using powerful viral promoters) do not make it possible to obtain such an efficiency in ES cells.

### Example 4

Production of ES Cells Allowing the Inducible Expression of a Transgene of Interest

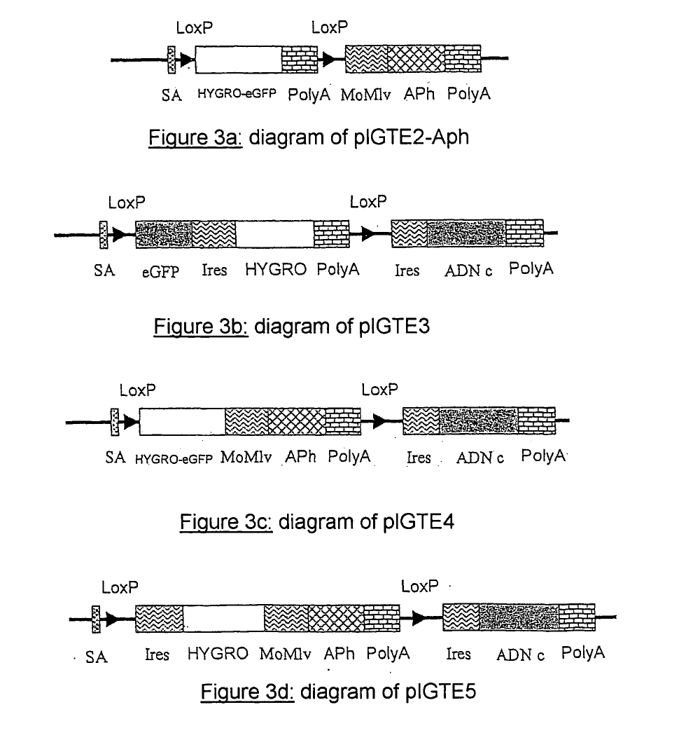

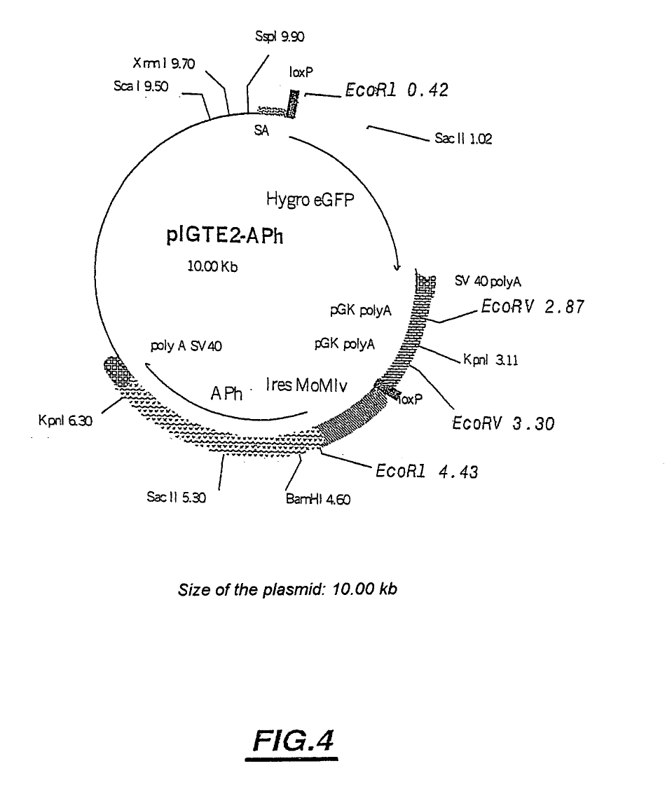

One of the uses of the ES-Cre-ER^{T2} cells is the production of a system for the inducible expression of transgenes in these cells. For that, the authors of the invention constructed another vector, called pIGTE2-Aph (cf FIG. 3A and FIG. 4), which comprises, as a transgene of interest, the Aph gene encoding human alkaline phosphatase. The gene can only be expressed if the vector is integrated near a powerful cellular promoter. On the other hand, its transcription is blocked by a loxP-STOP-loxP cassette which stops the synthesis of mRNA. In the presence of hydroxytamoxifen, the Cre-ER^{T2} recombinase is activated, the loxP-STOP-loxP cassette is excised and the Aph gene may be expressed. The authors of the invention isolated subclones of ES-Cre-ER^{T2} cells also containing a copy of this vector pIGTE2-Aph integrated into their genome. These subclones were brought into contact with 1 μM hydroxytamoxifen (OHT) in order to induce the expression of Aph. After 48 hours, the Aph activity was measured according to the Biolabs protocol (Ref. 172-1063).

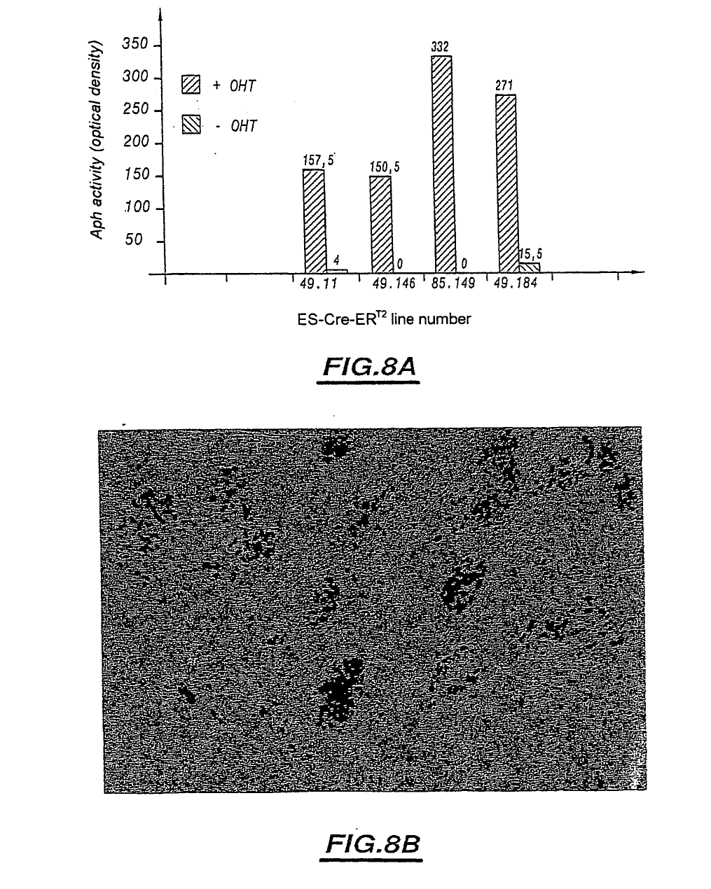

The authors of the invention were able to observe an expression of alkaline phosphatase closely dependent on the presence of hydroxytamoxifen in the culture medium. Thus, in some clones, the induction of the expression of alkaline phosphatase in the presence of hydroxytamoxifen reaches a factor of 80 while the background expression is zero (FIG. 8).

For comparison with the expression system of the invention, the authors also established, according to the protocol of Zhang et al., 1996, ES cell lines expressing Cre-ER^{T2} with the aid of a conventional expression system based on the pgk promoter of the gene encoding Phosphoglycerate Kinase (pgk-1). A vector for expression of β-galactosidase inducible by Cre whose function is also based on the pgk promoter was also introduced. Thus, in the presence of hydroxytamoxifen or of one of its derivatives, the expression of β-galactosidase is induced by Cre-ER^{T2}.

Various ES pgk Cre ER^{T2}/pgk β-galactosidase cell clones were brought into contact with hydroxytamoxifen (OHT) in order to induce expression of Aph. After 48 hours, the β-galactosidase activity was visualized by histochemical staining.

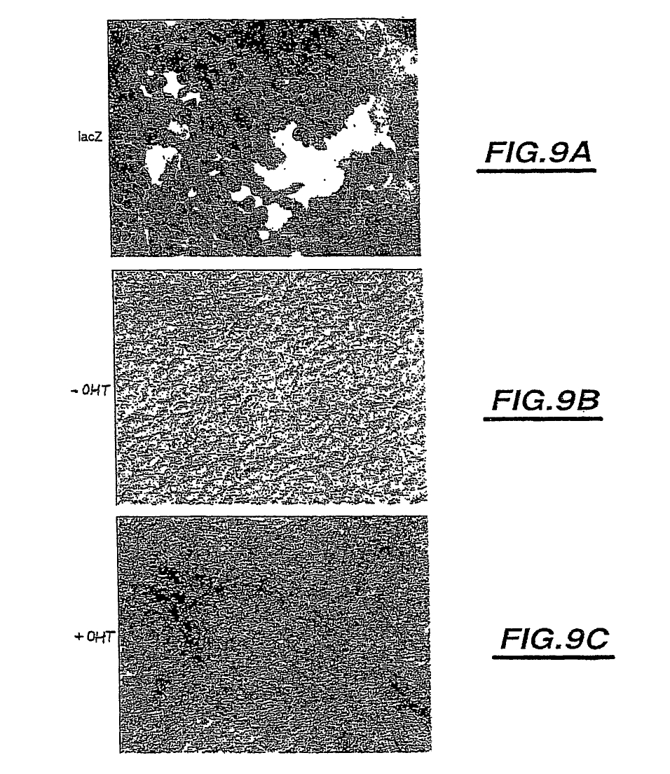

The best clone obtained is presented in FIG. 8B. It can be seen on this photograph that only a minority of cells express β-galactosidase (about 40% of the cells of the clone are stained blue). This result is far less than that obtained under the same conditions of induction with the ES-Cre ER^{T2}/pIGTE-Aph cell lines obtained using the technique according to the invention of “gene trap expression” (FIG. 9), 100% of the cells then being induced.

FIG. 9A represents an ES-Cre-ER^{T2}/pIGTE2-Aph line after histochemical staining in order to detect the β-galactosidase activity. The blue color comes from this activity. It can be observed that all the cells are very dark, which indicates a very high β-galactosidase activity and therefore a high expression of Cre-ER^{T2}.

FIG. 9B represents an ES-Cre-ER^{T2}/pIGTE2-Aph line cultured in the absence of hydroxytamoxifen. After histochemical staining in order to detect the alkaline phosphatase activity (Aph), no cell shows a black color characteristic of an Aph activity. There is therefore no induction of the expression of Aph in the absence of hydroxytamoxifen or of one of its derivatives.

FIG. 9C represents an ES-Cre-ER^{T2}/pIGTE2-Aph line cultured in the presence of 1 μM hydroxytamoxifen for 48 hours. After histochemical staining to detect the alkaline phosphatase (Aph) activity, 100% of the cells show a black color characteristic of an Aph activity. There is therefore induction of the expression of Aph in all the cells in the presence of an inducer.

These results are therefore considerably higher than those obtained with the other inducible expression systems in ES cells (Saez et al., 1997).

The authors of the invention then constructed vectors inducible by Cre-ER^{T2} whose function is based on that of pIGTE2-Aph. However, these vectors, designated pIGTE3, pIGTE4 and pIGTE5 (cf FIGS. 3B to 3D), were designed to inducibly overexpress proteins of interest other than Aph. Thus, the authors inserted into these vectors the sequences encoding cyclin D1 (pIGTE4-D1), cyclin D2 (pIGTE4-D2), proliferation inhibitors p18^{ink4c} (pIGTE4-p18^{ink4c}) and p21^{ciP1} (pIGTE4-p21^{ciP1}). After electroporation, the authors isolated subclones of ES-Cre ER^{T2} cells which have incorporated at least one copy of one of the vectors cited above. The authors thus established ES cell lines capable of inducibly overexpressing cyclin D1, cyclin D2, p18^{ink4c}, or p21^{cip1}.

The vector pIGTE2 Aph was constructed as follows: the vector ROSA β Geo (Friedrich et al., 1991) was digested with the enzymes SpeI and HindIII. The fragment of 300 bp thus obtained corresponds to the splice acceptor site. This fragment was inserted by cohesive ligation into the CMV Ires-Cre-ER^{T2} vector digested with SpeI and HindIII. The novel vector thus obtained is called pSA.

The vector pT102 was digested with the enzymes EcoRI and BSTEII and then self-religated. This operation made it possible to eliminate the PGK TK fragment from PT102. The novel vector thus obtained is called pT102-TK. This vector was digested with the enzyme NdeI. The fragment obtained corresponds to a cassette loxP-PGK Neo PolyA PolyA-loxP. The ends of this fragment were made blunt with T4 DNA polymerase. The fragment was then inserted by ligation into the vector pSA digested with the enzymes EcoRI and XhoI and whose ends had been made blunt with T4 DNA polymerase. The novel vector thus obtained is called pSA loxP-STOP-loxP.

The vector pHygEGFP (Clontech, catalog reference 6014-1) was digested with the enzyme BamHI. The 2 Kb fragment thus obtained corresponds to the sequence encoding the fusion protein HYGROeGFP. The ends of this fragment were made blunt using T4 DNA polymerase. The fragment was then inserted by ligation into the vector pSA loxP-STOP-loxP digested with the enzymes ApaI and NcoI and whose ends had been made blunt with T4 DNA polymerase. The novel vector thus obtained is called pIGTE2.

The vector PHW3 (Torrent et al., 1996) was digested with the enzymes SalI and SpeI. The fragment thus obtained corresponds to the Ires Aph sequence. The ends of this fragment were made blunt with T4 DNA polymerase. The fragment was then inserted by ligation into the vector pIresHyg (Clontech) digested with the enzyme XbaI and whose ends had been made blunt with T4 DNA polymerase. The novel vector thus obtained was called pIresHyg Ires Aph.

The vector pIresHyg Ires Aph was digested with the enzymes BglII and XhoI. The fragment thus obtained corresponds to the sequence Ires Aph polyA. The ends of this fragment were made blunt with T4 DNA polymerase. The fragment was then inserted by ligation into the vector pIGTE2 digested with the enzyme XbaI and whose ends had been made blunt with T4 DNA polymerase. The novel vector thus obtained was called pIGTE2 Aph.

The vector pIGTE3 was constructed as follows: the vector pEGFP-N1 (Clontech, catalog reference 6085-1) was digested with the enzymes BamHI and NotI. The 1 kb fragment thus obtained corresponds to the sequence encoding the eGFP protein. The ends of this fragment were made blunt using T4 DNA polymerase. The fragment was then inserted by ligation into the vector PIresHyg digested with the enzymes BstXI and NotI and whose ends had been made blunt with T4 DNA polymerase. The novel vector thus obtained is called pEGFP Ires HYGRO.

The vector pEGFP Ires HYGRO was digested with the enzymes BamHI and XbaI. The 3 kb fragment thus obtained corresponds to the sequence EGFP Ires HYGRO. The ends of this fragment were made blunt using T4 DNA polymerase. The fragment was then inserted by ligation into the vector pSA loxP-STOP-loxP digested with the enzymes ApaI and NcoI and whose ends had been made blunt with T4 DNA polymerase. The novel vector thus obtained is called pIGTE3.

The vector pIGTE4 was constructed as follows: the vector pSA loxP-STOP-loxP was digested with the enzymes XhoI and SpeI. The fragment thus obtained corresponds to the sequence SA loxP. This fragment was then inserted by ligation into the vector pIresHyg Ires Aph digested with the enzymes SpeI and HindIII. The noncohesive ends of the vector and of the insert were made blunt with T4 DNA polymerase. The novel vector thus obtained is called pSA HYGRO Ires Aph.

The vector pSA loxP-STOP-loxP was digested with the enzyme ClaI. The fragment thus obtained corresponds to the sequence polyApolyA-loxP. The ends of this fragment were made blunt with T4 DNA polymerase. The fragment was then inserted by ligation into the vector pSA HYGRO Ires Aph digested with the enzyme XhoI whose ends had been made blunt with T4 DNA polymerase. The novel vector thus obtained is called pIGTE4.

### Example 5

Production of Transgenic Mice from Selected ES-Cre-ER^{T2} Cell Lines

The ES-Cre-ER^{T2} cell lines which have integrated the vector pIGTE2-Aph into their genome were used to produce transgenic mice, by injection into blastocysts according to the protocol by Hogan et al., 1994.

The ES-Cre-ER^{T2}/pIGTE-Aph cells were aggregated to host embryos at the morula stage (Hogan et al., 1994, Manipulating the Mouse Embryo, Cold Spring Harbor Laboratory Press). The chimeric embryos thus obtained were reimplanted into a recipient mouse. The expression of Aph was then induced by an intraperitoneal injection of hydroxytamoxifen (1 mg) at 6.5 days post-coitus (dpc). After dissection at 8.5 dpc, the Aph activity was detected by histochemical staining. Thus, the cells which express Aph are stained brown while the cells which do not express Aph remain white.

An identical protocol was followed with the negative controls, with the difference that the injection at 6.5 dpc was carried out with a placebo.

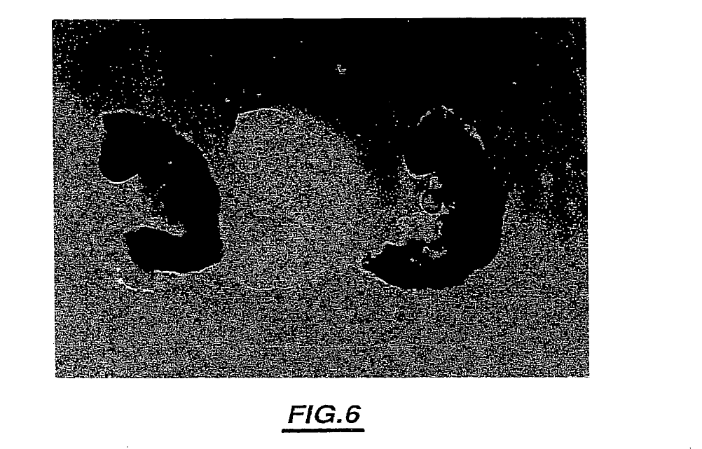

As can be seen in FIG. 6, the embryos induced in vivo with hydroxytamoxifen (on the right and on the left) strongly express Aph in all the tissues while the negative control (embryo at the center) expresses Aph only in a few cells.

### Example 6

Use of the Cre ER^{T2} Transgenic Mice According to the Invention in an Inducible Gene Invalidation System

6.1. Principle

The mice obtained in Example 5 are crossed with mice carrying a gene surrounded by two loxP sites (“floxed” gene). The floxed gene is functional because the loxP sites were introduced so as not to disrupt its function. Thus, “floxed” mice possess a wild-type phenotype.

In the second generation, 12.5% of the animals inherited the two alleles of the “floxed” gene and of the Cre ER^{T2} recombinase gene. This method makes it possible to induce the disappearance of a gene at any stage of development of mice. Hydroxytamoxifen is then injected by the intraperitoneal route in order to activate the recombinase and to induce the deletion of the gene.

6.2. Applications to the Study of Cancers

In humans, the deletion of the Rb-1 gene is involved in the appearance of several types of cancer with a high hereditary component, in particular retinoblastomas. In mice, the mutation of one allele of the Rb-1 gene only very slightly increases the frequency of the tumors. The mutation of the two alleles is by contrast lethal from the first embryonic stages. Using mice expressing the Cre-ER^{T2} recombinase, it is possible to induce the conditional inactivation of the two alleles of the Rb-1 gene in post-natal mice and to study the appearance of tumors in the various tissues. Other genes encoding tumor suppressor factors may be inactivated according to this protocol: the APC gene involved in colon cancers, the BRCA1 gene involved in breast and ovarian cancers.

6.3. Applications to the Study of Acute Pancreatitis

The p48 gene encodes a transcription factor necessary for the differentiation and the functioning of the exocrine pancreas. The inactivation of the p48 gene interrupts embryonic development. Using mice expressing the Cre-ER^{T2} recombinase, it is possible to induce the conditional inactivation of the two alleles of the p48 gene in adult mice, thus inducing pancreatic degeneration. These mice therefore constitute a model of acute pancreatitis.

### Example 7

Production of Differentiated Cells Useful for a Cell Transplant

7.1. Differentiation of ES-Cre-ER^{T2} Cells

The activity of the genes encoding the transcription factors pdx-1 and p48 appears to be necessary and sufficient for the induction of the differentiation of the endoderm into β type pancreatic cells. However, the constitutive overexpression of these genes in ES cells is not tolerated by the cell. The authors of the invention proposed introducing them in an “extinguished” configuration into the Cre-ER^{T2} cells, inducing differentiation into cells of the endoderm, and then activating the expression of the pdx-1 and p48 genes in order to promote pancreatic differentiation in vitro.

ES-Cre-ER^{T2} cell lines obtained in Example 4 comprising, as transgenes of interest whose expression is inducible by the Cre-ER^{T2} recombinase, the pdx-1 and p48 genes are therefore subjected to differentiation, according to a protocol reported by J. Odorico et al., Pancreatic gege expression in differentiating embryonic stem cell. Poster 324. Keystone symposia. Stem cells, asymmetric division and cell fate. Jan. 17-22, 2000, Keystone Colo. USA.

The embryonic stem cells are cultured in suspension in the presence of 5% CO₂ in a GMEM (Glasgow minimum essential medium) culture medium containing solely 10% fetal calf serum. These culture conditions induce the differentiation of ES cells into embryoid bodies. After 7 days, the embryoid bodies thus obtained are left to adhere and then cultured for 15 days, still in the same culture medium. A portion of the cells thus differentiated expresses markers specific for pancreatic cells such as pdx-1, insulin I, insulin II, glucagon or α-amylase.

7.2. Cell Transplant

These cells can be transplanted into the pancreas of animals, in a perspective of replacement cell therapy (Dinsmore et al., 1996).

### REFERENCES

Abremski et al., Studies on the properties of P1 site-specific recombination: evidence for topologically unlinked products following recombination. Cell 1983, 32: 1301-1311

Böger et al., (1999), Mechanisms of Development 83: 141-153

Brocard et al., A chimeric Cre recombinase inducible by synthetic, but not by natural ligands of the glucocorticoid receptor. Nucleic Acids Res. Sep. 1, 1998; 26(17): 4086-90

Dinsmore et al., Embryonic stem cells as a model for studying regulation of cellular differentiation. Theriogenology. Jan. 1, 1998; 49(1): 145-51

Dinsmore et al., Embryonic stem cells differentiated in vitro as a novel source of cells for transplantation. Cell Transplant. 1996 March-April; 5(2): 131-43

Feil et al., Ligand-activated site-specific recombination in mice, PNAS, 1996, vol. 93(20): 1887-1890

Feil et al., Regulation of Cre activity by mutated estrogen receptor ligand-binding domains. Biochem. Biophys. Res. Commun., Aug. 28, 1997; 237(3): 752-7

Fraichard et al., In vitro differentiation of embryonic stem cells into glial cells and functional neurons. J. Cell Sci. 1995 October; 108 (Pt 10): 3181-8

Friedrich et al., Promoter traps in embryonic stem cells: a genetic screen to identify and mutate developmental genes in mice. Genes Dev. 1991 September; 5(9): 1513-23

Gautier et al., Generation of small fusion genes carrying phleomycin resistance and Drosophila alcohol dehydrogenase reporter properties: their application in retroviral vectors. Exp. Cell Res. May 1, 1996; 224(2): 291-301

Hogan et al., (1994), Manipulating the Mouse Embryo, Cold Spring Harbor Laboratory Press

Jackson et al., (1990), The novel mechanism of initiation of picornavirus RNA translation, Trends Biochem. Sci., 15, 477-483

Kaminski et al., (1990), Initiation of encephalomyocarditis virus RNA translation: the authentic initiation site is not selected by a scanning mechanism, EMBO J, 9: 3753-3759

Kellendonk et al., Inducible site-specific recombination in the brain. J. Mol. Biol. Jan. 8, 1996; 285(1): 175-82

Logie C, Stewart AF. Ligand-regulated site-specific recombination. Proc. Natl. Acad. Sci. USA. Jun. 20, 1995; 92(13): 5940-4

Metzger et al., (1995), Proc. Natl. Acad. Sci., Conditional site-specific recombination in mammalian cells using a ligand-dependant chimeric Cre recombinase, 92: 6991-6995

Niwa et al., Efficient selection for high-expression transfectants with a novel eukaryotic vector. Gene. Dec. 15, 1991; 108(2): 193-9

Renoncourt et al., Neurons derived in vitro from ES cells express homeoproteins characteristic of motoneurons and interneurons. Mech. Dev. 1998 December; 79(1-2): 185-97

Reubinoff et al., Embryonic stem cell lines from human blastocysts: somatic differentiation in vitro. Nat. Biotechnol. 2000 April; 18(4): 399-404

Saez et al., (1997), Current opinion in Biotechnology, Inducible gene expression in mammalian cells as transgenic mice, 8: 608-616

Sambrook et al., (1989) Molecular cloning, a laboratory Manual, Cold Spring Harbor Laboratory Press

Takahashi et al., Characterization of hematopoietic lineage-specific gene expression by ES cell in vitro differentiation induction system. Blood. Feb. 1, 2000; 95(3): 870-8

Torrent et al., Stable MLV-VL30 dicistronic retroviral vectors with a VL30 or MOMLV sequence promoting both packaging of genomic RNA and expression of the 3′ cistron. Hum. Gene Ther. Mar. 20, 1996; 7(5): 603-12

Watt Fiona and Hogan Brigid (2000), Science, 287: 1427-1430

Zhang et al., (1996), Nucleic Acids Research, vol. 24, No. 4, 543-548

1. A nucleic acid construct comprising
i) a splice acceptor site at the 5′ position,
ii) a selection sequence, optionally preceded upstream by a sequence allowing its translation by the ribosomes,
iii) a sequence encoding a protein of interest distinct from the selection sequence, said coding sequence being preceded upstream by a sequence allowing its translation by the ribosomes,
iv) a transcription termination sequence at the 3′ position,
said construct being free of any promoter for transcription of said selection sequence or of said sequence encoding a protein of interest,
it being in addition understood that said protein of interest is not the transactivator protein tTA.

2. The nucleic acid construct as claimed in claim 1, in which said sequence encoding a protein of interest is a sequence encoding an inducible recombinase.

3. The nucleic acid construct as claimed in claim 2, in which said sequence encoding a protein of interest is a sequence encoding the CRE recombinase modified so as to be inducible by tamoxifen, said sequence being designated Cre-ER^{T2}.

4. The nucleic acid construct as claimed in claim 2, comprising, from upstream to downstream,
i) a splice acceptor site,
ii) a neomycin resistance selection sequence, preceded upstream by a detectable sequence, encoding β-galactosidase, the whole being designated β-geo,
iii) a Cre-ER^{T2} sequence, preceded upstream by an IRES sequence allowing its translation by the ribosomes,
iv) at least one polyA sequence containing at least one STOP, for termination of transcription.

5. The nucleic acid construct as claimed in claim 1, in which said sequence encoding a protein of interest is a sequence encoding a protein of therapeutic interest or a differentiation factor.

6. The nucleic acid construct as claimed in claim 1, in which said sequence encoding a protein of interest is replaced by an antisense sequence.

7. The nucleic acid construct as claimed in claim 1, in which recombinase recognition sequences such as the LoxP sequences surround the cassette formed by said selection sequence, optionally preceded upstream by at least one sequence allowing its translation by the ribosomes, and followed downstream by an additional transcription termination sequence, said cassette being placed upstream of said sequence encoding the protein of interest.

8. The nucleic acid construct as claimed in claim 7, comprising, from upstream to downstream,
i) a splice acceptor site,
ii) a cassette formed from upstream to downstream by
optionally one sequence, such as an IRES sequence, allowing translation, by the ribosomes, of the selection sequence which follows,
a selection sequence, such as a sequence for resistance to hygromycin,
optionally a sequence encoding a detectable marker protein, such as a sequence encoding human alkaline phosphatase (Aph), preceded by a sequence, allowing its translation by the ribosomes,
a transcription termination sequence comprising several STOP sites in several polyAs,
said cassette being surrounded by LoxP sequences,
iii) a sequence encoding a protein of interest, said coding sequence being preceded upstream by a sequence, such as an IRES sequence, allowing its translation by the ribosomes,
iv) a transcription termination sequence.

9. A vector into which is inserted a nucleic acid construct as claimed in one of the preceding claims.

10. A host cell into which at least one vector as claimed in claim 9 has been stably transferred.

11. The cell as claimed in claim 10, into whose genome are cointegrated at least one nucleic acid construct as claimed in one of claims 2 to 4 comprising a sequence encoding an inducible recombinase, and at least one nucleic acid construct as claimed in either of claims 7 and 8, comprising sequences for recognition of said recombinase and a sequence encoding a protein of interest.

12. The cell as claimed in either of claims 10 and 11, which is an embryonic stem cell (ES cell) or an embryonic germ stem cell (EG cell).

13. The cell as claimed in claim 12, which is an ES cell or an EG cell of a nonhuman animal, such as in particular a mouse.

14. A cell bank comprising cell lines as claimed in one of claims 10 to 13 to whose genome said vector(s) has (have) become specifically integrated.

15. A nonhuman transgenic animal, such as in particular a mouse, which is capable of being obtained from a stem cell as claimed in claim 13.

16. A method for preparing differentiated cells, in which totipotent cells as claimed in claim 12 are cultured in the presence of differentiation agents and, where appropriate, a recombinase inducing agent.

17. A differentiated cell which can be obtained by the method of claim 16, and useful in particular for a cell transplant.

18. An in vitro method for producing a recombinant protein of interest, in which there are cultured cells as claimed in one of claims 10 to 13 or 17, into whose genome there has been integrated a nucleic acid construct comprising a sequence encoding a recombinant protein of interest, under conditions allowing the expression of said protein of interest, and the protein thus produced is recovered.

19. The method as claimed in claim 18, in which said cells are embryonic stem cells, into whose genome there are cointegrated at least one nucleic acid construct as claimed in one of claims 2 to 4 comprising a sequence encoding an inducible recombinase and at least one nucleic acid construct as claimed in either of claims 7 and 8, comprising sequences for recognition of said recombinase, and a sequence encoding a protein of interest,
said cells being cultured in the presence of differentiation agents, the differentiated cells thus obtained then being brought into contact with an agent inducing said recombinase, so as to allow the expression of said protein of interest.

20. A method for producing a nonhuman transgenic animal which is in particular useful as a model for studying genes involved in a pathology, in which (a) at least one nucleic acid construct as claimed in one of claims 2 to 4 comprising a sequence encoding an inducible recombinase is integrated into the genome of a nonhuman animal, and (b) the nonhuman animal thus obtained is crossed with a nonhuman animal in whose genome a gene of interest is surrounded by two sites recognized by the inducible recombinase, so as to obtain a nonhuman transgenic animal which, when it is subjected to an agent inducing said recombinase, undergoes deletion of said gene of interest.

21. A nonhuman transgenic animal, such as in particular a mouse, which can be obtained by the method as claimed in claim 20.


SEQUENCE LISTING (2 sequences)

SEQ ID NO: 1 - DNA, 31 bp, Artificial sequence
  Artificial sequence description PCR primer
agaaccaatgcatgctgatcagcgaggttta

SEQ ID NO: 2 - DNA, 38 bp, Artificial sequence
  Artificial sequence description PCR primer
aaggaaaaaagggggcgcctatggctcgtactctatag